(19) United States
(12) Patent Application Publication
(10) Pub. No.: US 2025/0134520 A1
(43) Pub. Date: May 1, 2025

(54) DEVICES FOR SECURING IMPLANTS TO BONE TISSUE

(71) Applicant: 4WEB, LLC, Dallas, TX (US)

(72) Inventors: Jessee Hunt, Plano, TX (US); David Knox, Plano, TX (US)

(21) Appl. No.: 18/927,007

(22) Filed: Oct. 25, 2024

Related U.S. Application Data
(60) Provisional application No. 63/593,801, filed on Oct. 27, 2023.

(51) Int. Cl. A61B 17/064; A61B 17/04

(52) U.S. Cl. CPC A61B 17/064; A61B 2017/0412

(57) ABSTRACT

Various examples of implant systems and related apparatus, and methods of operating the same are described herein. In various embodiments, a securing device is coupled to an implant. The securing device may be used to secure the implant to human tissue (e.g., bone tissue) after the implant is positioned on the tissue. The securing device may include anchor devices that move through the body of the implant and penetrate the human tissue. With the anchor devices moved through the implant, first ends of the anchor devices may engage with the human tissue while second ends of the anchor devices engage the implant body, thereby securing the implant to the human tissue.

### PRIORITY CLAIM

The present application claims priority to U.S. Provisional App. No. 63/593,801, entitled “DEVICES FOR SECURING IMPLANTS TO BONE TISSUE,” filed Oct. 27, 2023, the disclosure of which is incorporated by reference herein in its entirety.

### BACKGROUND OF THE INVENTION

### 1. Field of the Invention

The present invention relates generally to medical devices and, more specifically, to implants and devices for securing implants to human bone tissue.

### 2. Description of the Relevant Art

Implants may be used in human and/or animals to support and/or secure one or more bones. For example, implants may be used in the spine to support and/or replace damaged tissue between the vertebrae in the spine. Once implanted between two vertebrae, the implant may provide support between the two vertebrae and bone growth may take place around and through the implant to at least partially fuse the two vertebrae for long-term support. Implants may include relatively large rims with solid material that may cover, for example, 50% of the area that interacts with the endplate. The rim may provide a contact area between the implant and the vertebral endplates. Large rims may have several drawbacks. For example, large rims may impede bone growth and reduce the size of the bone column fusing the superior and inferior vertebral bodies. Additionally, large rims preferentially support and regionalize loads, preventing distribution of force and accommodating response. The process of localizing loading also serves to under load other areas of the vertebral bodies, thereby activating regional resorption according to negative microstrain.

Spinal implants may include open channels through the center of the supporting rims in a superior/inferior direction. The open channel design may require members of the implant that separate the rims that interact with the vertebral endplates to absorb the compressive forces between the vertebral endplates. This may increase the pressure on smaller areas of the vertebral endplates and may potentially lead to stress risers in the vertebral endplates. Further, while bone graft material is often used in conjunction with implants to encourage bone growth, the open column design of implants may reduce the likelihood of bone graft material from securing itself to the implant which could result in a bio-mechanical cooperation that is not conducive to promoting good fusion.

Bone graft material may be packed into the implant in a high-pressure state to prevent bone graft material from exiting the implant while being placed between the vertebral endplates. The high-pressure state may also reduce the potential for the bone graft material loosening due to motion between the implant and the vertebral endplates or compressive forces experienced during settling of the implant. In addition, a high-pressure environment may allow the bone graft material to re-model and fuse at greater strength. High-pressure states, however, may be difficult to create and maintain for the bone graft material in an implant. In particular, the lack of attachment of the bulk graft cannot fully accept or integrate the differential loading anticipated in normal kinetic scope.

### BRIEF DESCRIPTION OF THE DRAWINGS

Advantages of the present invention will become apparent to those skilled in the art with the benefit of the following detailed description of embodiments and upon reference to the accompanying drawings in which:

FIGS. 1A-1B illustrate views of an implant with lordosis, according to an embodiment.

FIGS. 2A-2D illustrate views of an implant without lordosis, according to an embodiment.

FIGS. 3A-3C illustrate progressive sectioned views of the implant showing the internal structure of the implant, according to an embodiment.

FIG. 3D illustrates an isometric view of the implant, according to an embodiment.

FIGS. 4A-4D illustrate another configuration of the web structure, according to an embodiment.

FIGS. 5A and 5B depict isometric perspective view representations of a contemplated embodiment of an implant and a securing device.

FIGS. 6A-6C depict cross-sectional side view representations of the contemplated embodiment of an implant and a securing device.

FIGS. 7A-7C depict top view representations of the contemplated embodiment of an implant and a securing device.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims. Note, the headings are for organizational purposes only and are not meant to be used to limit or interpret the description or claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

As used herein a “truss structure” is a structure having one or more elongate struts connected at joints referred to as nodes. Trusses may include variants of a pratt truss, king post truss, queen post truss, town's lattice truss, planar truss, space truss, and/or a vierendeel truss (other trusses may also be used). A “truss unit” is a structure having a perimeter defined by three or more elongate struts.”

As used herein a “planar truss” is a truss structure where all of the struts and nodes lie substantially within a single two-dimensional plane. A planar truss, for example, may include one or more “truss units” where each of the struts is a substantially straight member such that the entirety of the struts and the nodes of the one or more truss units lie in substantially the same plane. A truss unit where each of the struts is a substantially straight strut and the entirety of the struts and the nodes of the truss unit lie in substantially the same plane is referred to as a “planar truss unit.”

As used herein a “space truss” is a truss having struts and nodes that are not substantially confined in a single two-dimensional plane. A space truss may include two or more planar trusses (e.g., planar truss units) wherein at least one of the two or more planar trusses lies in a plane that is not substantially parallel to a plane of at least one or more of the other two or more planar trusses. A space truss, for example, may include two planar truss units adjacent to one another (e.g., sharing a common strut) wherein each of the planar truss units lie in separate planes that are angled with respect to one another (e.g., not parallel to one another).

As used herein a “triangular truss” is a structure having one or more triangular units that are formed by three straight struts connected at joints referred to as nodes. For example, a triangular truss may include three straight elongate strut members that are coupled to one another at three nodes to from a triangular shaped truss. As used herein a “planar triangular truss” is a triangular truss structure where all of the struts and nodes lie substantially within a single two-dimensional plane. Each triangular unit may be referred to as a “triangular truss unit.” A triangular truss unit where each of the struts is a substantially straight member such that the entirety of the struts and the nodes of the triangular truss units lie in substantially the same plane is referred to as a “planar triangular truss unit.” As used herein a “triangular space truss” is a space truss including one or more triangular truss units.

In accordance with the descriptions herein, in various embodiments, an implant may include a web structure. The web structure for the implant may include a micro truss design. In some embodiments, the micro truss design may include a web structure with multiple struts. Other web structures are also contemplated. The web structure may extend throughout the implant (including a central portion of the implant). The web structure may thus reinforce the implant along multiple planes (including internal implant load bearing) and provide increased area for bone graft fusion. The web structure may be used in implants such as spinal implants, corpectomy devices, hip replacements, knee replacements, long bone reconstruction scaffolding, and cranio-maxillofacial implants foot and ankle, hand and wrist, shoulder and elbow (large joint, small joint, extremities). Other implant uses are also contemplated. In some embodiments, the web structure for the implant may include one or more geometric objects (e.g., polyhedrons). In some embodiments, the web structure may not include a pattern of geometrical building blocks (e.g., an irregular pattern of struts may be used in the implant). In some embodiments, the web structure may include a triangulated web structure including two or more tetrahedrons. A tetrahedron may include four triangular faces in which three of the four triangles meet at each vertex. The web structure may further include two tetrahedrons placed together at two adjacent faces to form a web structure with a hexahedron-shaped frame (including six faces). In some embodiments, multiple hexahedron-shaped web structures may be arranged in a side-by-side manner. The web structures may connect directly through side vertices (e.g., two or more hexahedron-shaped web structures may share a vertex). In some embodiments, the web structure may be angled to provide lordosis to the implant.

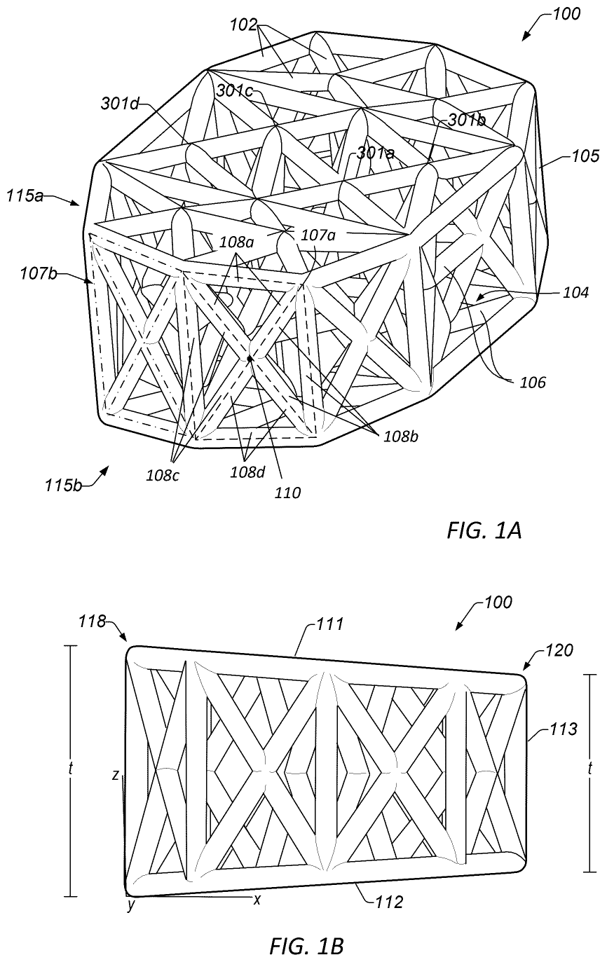

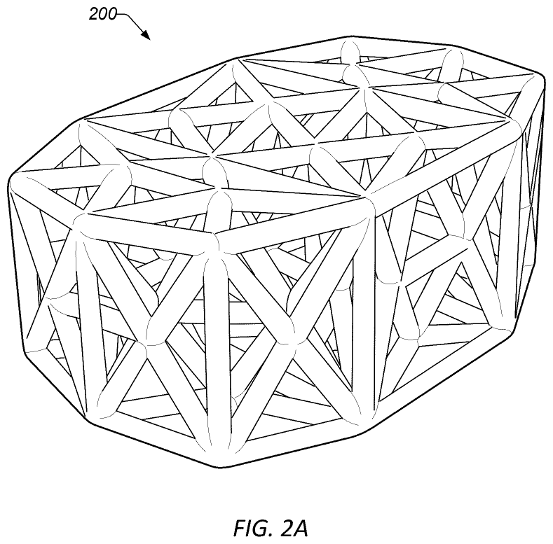

FIGS. 1A-1B illustrate views of implant 100, according to an embodiment. The specifically depicted implant 100 may be used, for example, in anterior lumbar inter-body fusion (ALIF) or posterior lumbar inter-body fusion (PLIF), however, it should be understood that implant 100 nay have a variety of shapes suitable for bone fusion applications. In some embodiments, implant 100 may include a web structure with one or more trusses 102 (e.g., planar and space trusses). Implant 100 may be used in various types of implants for humans or animals such as spinal implants, corpectomy devices, knee replacements, hip replacements, long bone reconstruction scaffolding, and cranio-maxillofacial implants, foot and ankle, hand and wrist, shoulder and elbow (large joint, small joint, extremity as well as custom trauma implants). Other implant uses are also contemplated.

In various embodiments, the trusses 102 of the web structure may include one or more planar truss units (e.g., planar triangular truss units) constructed with straight or curved/arched members (e.g., struts) connected at various nodes. In some embodiments, the trusses 102 may be micro-trusses. A “micro-truss” is a truss having dimensions sufficiently small enough such that a plurality of micro-trusses can be assembled or otherwise coupled to one another to form a web structure having a small enough overall dimension (e.g., height, length and width) such that substantially all of the web structure can be inserted into an implant location (e.g., between two vertebra). Such a web structure and its micro-trusses can thus be employed to receive and distribute throughout the web structure loading forces of the surrounding tissue (e.g., vertebra, bone, or the like). In one embodiment, the diameters of the struts forming the micro-truss may be between about 0.25 millimeters (mm) and 5 mm in diameter (e.g., a diameter of about 0.25 mm, 0.5 mm, 0.6 mm, 0.7 mm, 0.8 mm, 0.9 mm, 1 mm, 2 mm, 3 mm, 4 mm, or 5 mm). In one embodiment, a micro-truss may have an overall length or width of less than about 1 inch (e.g., a length less than about 0.9 in, 0.8 in, 0.7 in, 0.6 in, 0.5 in, 0.4 in, 0.3 in, 0.2 in, 0.1 in).

As depicted, for example, in FIGS. 1A-1B, the web structure may extend throughout implant 100 (including the central portion of implant 100) to provide support throughout implant 100. Trusses 102 of implant 100 may thus support implant 100 against tensile, compressive, and shear forces. Web structure may also reinforce implant 100 along multiple planes. The external truss structure may, for example, provide support against tensile and compressive forces acting vertically through the implant, and the internal web structure may provide support against tensile, compressive, and shear forces along the various planes containing the respective trusses. In some embodiments, the web structure includes trusses 102 that form a triangulated web structure with multiple struts (e.g., struts 103a-f) (struts are generally referred to herein as “struts 103”).

In one embodiment, web structure of the implant 100 may include an internal web structure that is at least partially enclosed by an external truss structure. For example, in one embodiment, web structure 101 may include an internal web structure that includes a space truss having at least a portion of the space truss surrounded by an external truss structure that includes one or more planar trusses formed with a plurality of planar truss units that lie substantially in a single plane. FIG. 1A depicts an embodiment of implant 100 having an internal web structure 104 and an external truss structure 105. In the illustrated embodiment, internal web structure 104 includes a space truss defined by a plurality of planar truss units 106 coupled at an angle with respect to one another such that each adjacent truss unit is not co-planar with each adjacent truss units. Adjacent truss units may include two truss units that share a strut and the respective two nodes at the ends of the shared strut.

In one embodiment, external truss structure 105 includes a plurality of planar trusses that are coupled about an exterior, interior or other portion of the implant. For example, in the illustrated embodiment, the external truss structure 105 includes a series of planar trusses 107a,b that are coupled to one another. Planar truss 107a is denoted by a dashed line [- - -],planar truss 107b is denoted by dotted-dashed line [- - -]. Each planar truss is formed from a plurality of planar truss units (e.g., triangular planar truss units. As depicted, planar truss 107a includes four triangular planar truss units 108a,b,c,d having a common vertex 110 and arranged to form a generally rectangular structure that lies in a single common plane. In other words, the four triangular planar truss units are arranged to form a substantially rectangular structure having “X” shaped struts extend from one corner of the rectangular structure to the opposite corner of the rectangular structure. As depicted, the substantially rectangular structure may include a trapezoidal shape. As described in more detail below, the trapezoidal shape may be conducive to providing an implant including lordosis. Lordosis may include an angled orientation of surfaces (e.g., top and bottom) of an implant that provides for differences in thickness in anterior and posterior regions of the implant such that the implant is conducive for supporting the curvature of a vertebral column.

In one embodiment, the planar trusses that form the external truss are coupled to one another, and are aligned along at least one axis. For example, in FIG. 1A, planar truss section 107a is coupled to an adjacent planar truss 107b. Planer truss sections 107a,b are not parallel in all directions. Planar truss sections 107a,b are, however, arranged parallel to one another in at least one direction (e.g., the vertical direction between the top and the bottom faces of implant 100). For example, planar trusses 107a,b and the additional planar trusses are arranged in series with an angle relative to one another to form a generally circular or polygon shaped enclosure having substantially vertical walls defined by the planar trusses and the planar truss units arranged in the vertical direction.

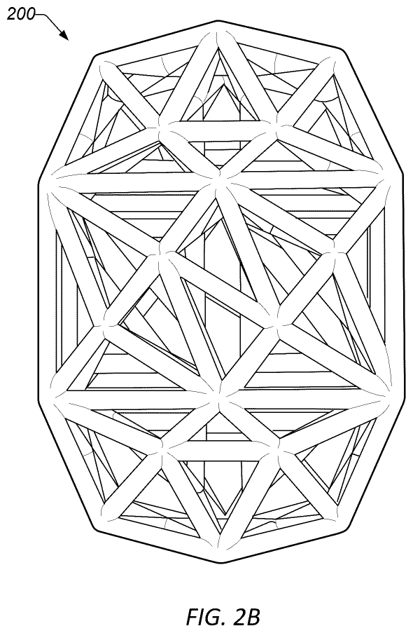

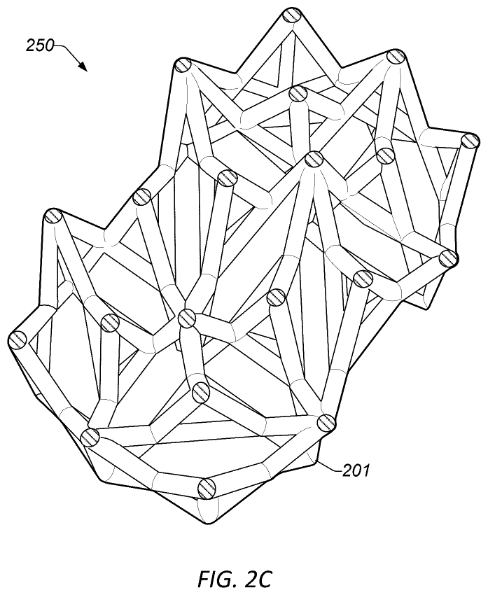

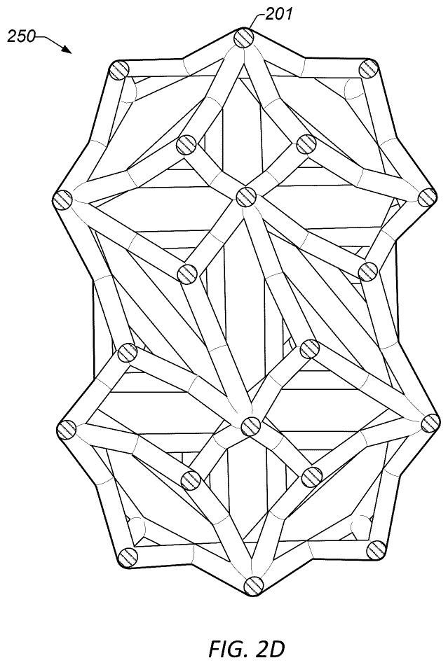

In one embodiment, the external truss portion may encompass the sides, top, and/or bottom of the implant. For example, in one embodiment, the external truss portion may include a top region, side regions, and/or a bottom region. FIG. 1A depicts an embodiment of implant 100 wherein external truss portion 105 includes a top 111, bottom 112 and a side region 113. As described above, side region 113 includes a series of planar trusses arranged vertically to form a circular/polygon ring-like structure that completely or at least partially surrounds the perimeter of the space truss disposed in the central portion of implant 100. In the depicted embodiment, top portion 111 of external truss structure 105 includes a plurality of truss units coupled to one another to form a planar truss that cover substantially all of the top region of internal web structure 104. In the illustrated embodiment, the top portion 111 spans entirely the region between top edges of the side portion 113 of external truss structure 105. In the illustrated embodiment, top portion 111 is formed from a single planar truss that includes a plurality of truss units that lie in substantially the same plane. In other words, the planar truss of top portion 111 defines a generally flat surface. Although difficult to view in FIG. 1, the underside of implant 100 may include the bottom portion 112 having a configuration similar to that of the top portion 111. In other embodiments, external truss structure 105 may include a partial side, top and/or bottom external truss portions. Or may not include one or more of the side, top and bottom external truss portions. For example, as described in more detail below, FIG. 2C depicts an embodiment of implant 100 that includes an internal web structure formed from space trusses, that does not have an external truss structure.

In some embodiments, implant 100 may be formed from a biocompatible material such as a titanium alloy (e.g., γTitanium Aluminides), cobalt, chromium, stainless steel, Polyetheretherketone (PEEK), ceramics, etc. Other materials are also contemplated. In some embodiments, implant 100 may be made through a rapid prototyping process (e.g., electron beam melting (EBM) process) as further described below. Other processes are also possible (e.g., injection molding, casting, sintering, selective laser sintering (SLS), Direct Metal Laser Sintering (DMLS), etc). SLS may include laser-sintering of high-performance polymers such as that provided by EOS of North America, Inc., headquartered in Novi, Michigan, U.S.A. High-performance polymers may include various forms of PEEK (e.g., HP3 having a tensile strength of up to about 95 mega Pascal (MPa) and a Young's modulus of up to about 4400 MPa and continuous operating temperature between about 180° C. (356° F.) and 260° C. (500° F.)). Other materials may include PA 12 and PA 11 provided by EOS of North America, Inc.

As described above, in some embodiments the web structure may be formed from a plurality of triangular planar truss units. In some embodiments, the planar truss units may be coupled to each other to define polyhedrons that define the internal web structure. Examples of polyhedron structures that may be created by joining planar truss units include, but are not limited to, tetrahedrons, pentahedrons, hexahedrons, heptahedrons, pyramids, octahedrons, dodecahedrons, icosahedrons, and spherical fullerenes. In some embodiments, such as those described above, the space truss of the web structure may connect multiple midpoints of tetrahedron building blocks and include a regular pattern of tetrahedron blocks arranged adjacent one another. In some embodiments, the web structure may not include a pattern of geometrical building blocks. Examples of implants composed of a web structure are described in U.S. Published Patent Applications Nos.: 2010/0161061; 2011/0196495; 20110313532; and 2013/0030529, each of which is incorporated herein by reference.

As shown in FIG. 1A, top surface 115a and bottom surface 115b of implant 100 may include triangles, squares, circles or other shapes (e.g., a random or custom design). Top and bottom surfaces 115a,b may be used to connect the top and bottom vertices of various geometrical building blocks used in the web structure of implant 100. For example, each vertex may be connected through struts to the neighboring vertices of other geometrical building blocks. Top surface 115a may include other strut networks and/or connections. In some embodiments, bottom surface 115b may mirror the top surface (and/or have other designs). In some embodiments, top surface 115a and bottom surface 115b may engage respective surfaces of two adjacent vertebrae when implant 100 is implanted.

As depicted in FIG. 1B, implant 100 may include lordosis (e.g., an angle in top and/or bottom surfaces 115a,b approximately in a range of 4 to 15 degrees (such as 4, 5, 6, 7, 8, 9, 10, 11, 12, 13, 14, or 15 degrees)) to further support the adjacent vertebrae when implanted. As described above, lordosis may include an angled orientation of surfaces (e.g., top and bottom) that provide for differences in thickness in the anterior and posterior portions of the implant such that the implant is conducive for supporting the curvature of a vertebral column. In the illustrated embodiment, the thickness of implant 100 is greater at or near the anterior portion 118 and lesser at or near the posterior portion 120 of the implant. In the illustrated embodiment, the side portions of external truss structure are arranged substantially vertically, and the lordosis is formed by the angles of the top portion 111 and bottom portion 112 of external truss structure. For example, in the illustrated embodiment, top portion 111 and bottom portion 112 of external truss structure are not perpendicular to the vertical plane defined by the side portion 113. Rather, the top portion 111 and bottom portion 112 are arranged with an acute angle relative to the vertical plane of side portion 113 at or near the anterior region 118 of implant 100 and with an obtuse angle relative to the vertical plane of side portion 113 at or near posterior region 120 of implant 100. As depicted, the vertical struts that form the planar truss of side portion 113 of external truss structure proximate posterior region 120 of implant 100 are shorter than struts that form side portion of external truss structure proximate anterior region 118 of implant 100. In the illustrated embodiment, in which the vertical trusses are substantially evenly spaced, the struts forming the “X” cross members of the side planar trusses proximate the posterior region 120 of implant 100 are shorter than struts forming the “X” cross members of the side planar trusses proximate the anterior region 118 of implant 100. Other embodiments may include variations in the arrangement of the trusses to provide various configurations of the implant. For example, in some embodiments only one or neither of the top and bottom external truss portions may be non-perpendicular to the side portions of the external truss proximate the anterior and posterior portions of the implant. Further, the side, top, and/or bottom portions may include multiple planar trusses angled relative to one another in any orientation. For example, the top or bottom portions may include four planar trusses, each formed of multiple truss units, such that the portion(s) includes a pyramidal like shape.

In some embodiments, the implant may not include lordosis. For example, FIGS. 2A-2B illustrate two views of an embodiment of an implant 200 without lordosis. In some embodiments, the top surface and bottom surface may not include connecting struts. For example, FIGS. 2C-2D illustrate two views of implant 250 without outer struts (e.g., without external truss portions formed of planar trusses). In the illustrated embodiment, implant 250 includes an internal web structure and does not include an external truss structure. For example, in the illustrated embodiment, the exterior faces of implant 250 are defined by a plurality of truss units that are angled relative to each of its adjacent truss units. The relative alignment of the truss units results in a non-planar exterior that includes a plurality of pointed junctions. The pointed junctions (e.g., pointed junction 201) may operate to dig into the surrounding bone to hold the implant in place (for example, if the implant is being used in a corpectomy device).

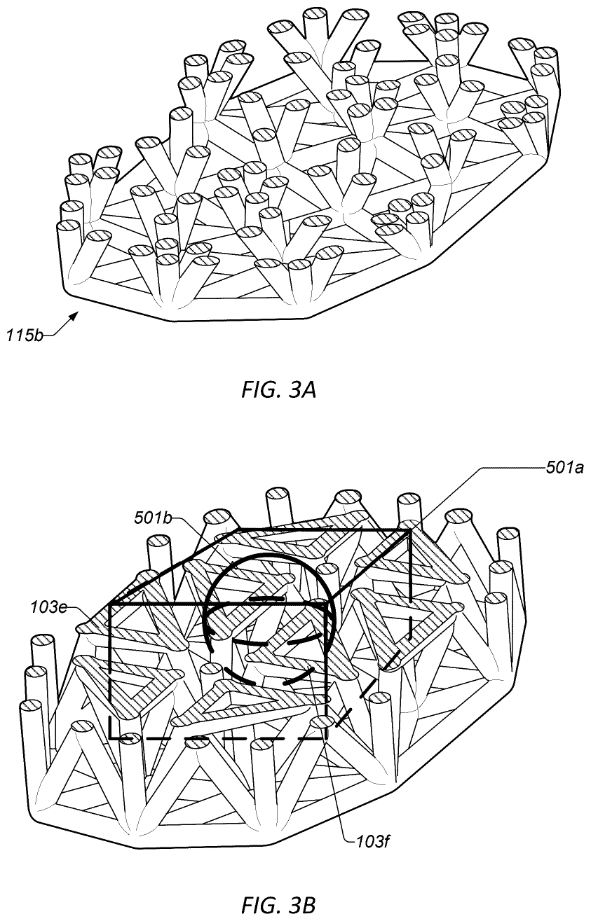

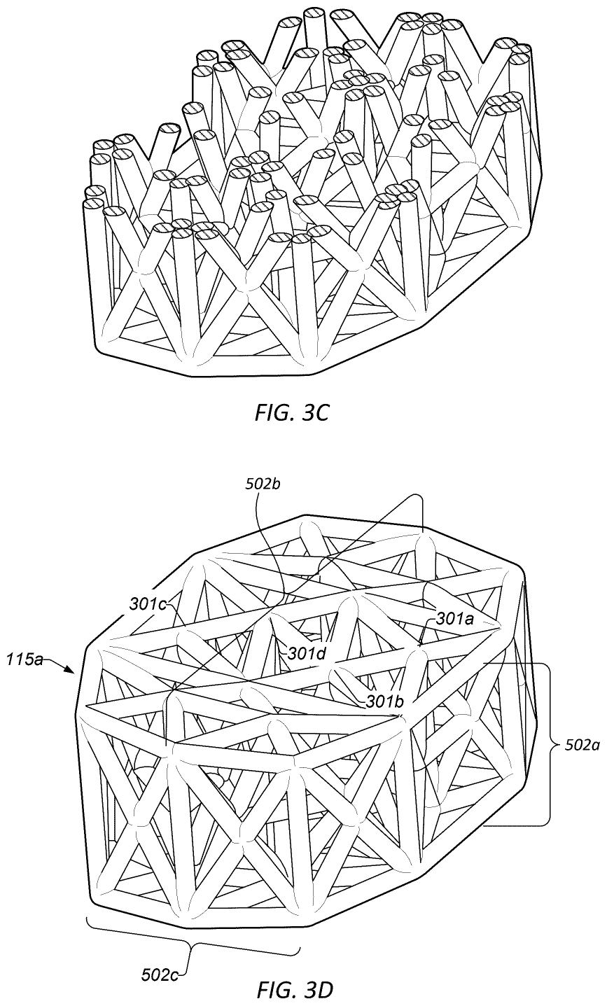

FIGS. 3A-3C illustrate progressive sectioned views of implant 100 showing the internal structure of implant 100, according to an embodiment. FIG. 3A illustrates a sectioned view of a lower portion of implant 100. Bottom surface 115b is shown with various struts (e.g., struts 103) extending upward from bottom surface 115b. FIG. 3B illustrates a sectioned view approximately mid-way through implant 100. Struts, such as struts 103e,f, shared by various stacked tetrahedrons in the web structure are shown. Some struts extend through central portion 501a and/or 501b of implant 100. FIG. 3B also shows central portions 501a,b of implant 100. In some embodiments, central portion 501a may include a rectangular region that has a width of approximately 50% of the implant width, a height of approximately 50% of the implant height, and a length of approximately 50% of the implant length and located in the center of implant 100. In some embodiments, central portion 501b may encompass a region (e.g., a spherical region, square region, etc.) of approximately a radius of approximately ⅛ to ¼ of the width of implant 100 around a position located approximately at one half the width, approximately one half the length, and approximately one-half the height of implant 100 (i.e., the center of implant 100). Other central portions are also contemplated. For example, the central portion may include a square region with a length of one of the sides of the square region approximately ¼ to ½ the width of implant 100 around a position approximately at one half the width, approximately one half the length, and approximately one half the height of the implant. An example height 502a, width 502b, and length 502c, is shown in FIG. 3D. In some embodiments, the height may be up to about 75 mm or more. In some embodiments, such as those used for long bone reconstruction, the width and/or length could be approximately 7 inches or longer. In some embodiments, the width, length, and/or height may vary along implant 100 (e.g., the height may vary if the implant includes lordosis). The height may be taken at one of the opposing sides, the middle, and/or may be an average of one or more heights along the length of implant 100. The web structure may extend through central portion 501a,b of the implant (e.g., at least one strut of the web structure may pass at least partially through central portion 501a,b). FIG. 3C illustrates another sectioned view showing sectioned views of top tetrahedrons in the web structure. FIG. 3D shows a complete view of implant 100 including top surface 115a with vertices 301a-d.

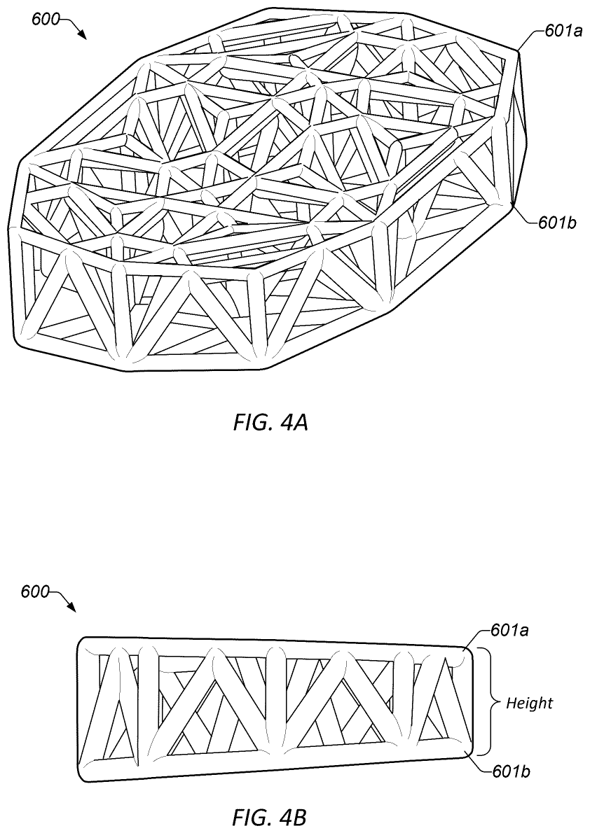

FIGS. 4A-4D illustrate alternate embodiments of an implant. In some embodiments, different sections of the hexahedron-shaped geometric design may be used. For example, as seen in FIG. 4A, the bottom half of the hexahedron-shaped geometric design may be used (primarily including the lower tetrahedron structures). If using the bottom half of the design, implant 600 may be expanded proportionately to have similar overall dimensions as the hexahedron-shaped geometric design (e.g., the tetrahedrons may be expanded to approximately twice the height of the tetrahedrons in the hexahedron-shaped geometric design to give implant 600 a height approximately the same as the hexahedron-shaped geometric design). In some embodiments, implant 600 may also be angled (e.g., on top surface 601a and/or bottom surface 601b) to provide implant 600 with lordosis to, in some embodiments, have a better fit between the vertebral endplates. Top surface 601a and/or bottom surface 601b may also include struts to connect nodes of implant 600 (e.g., see the strut network on the top surface in FIG. 4A). Other patterns of struts for top surface 601a and/or bottom surface 601b may also be used. In some embodiments, implant 600 may not include negative angles between struts and may thus be easier to create through a casting or molding process.

FIGS. 4C-4D illustrate another alternate embodiment of an implant. In some embodiments, approximately the middle 40 to 60 percent of the hexahedron-shaped geometric design may be used in implant 650. For example, if an overall height of the hexahedron-shaped geometric design is approximately 37 mm, approximately the bottom 10 mm and approximately the top 10 mm of the design may be removed and approximately the middle 17 mm of the design may be used for the implant. Middle portion of implant 650 may then be expanded proportionately such that the approximate height of the expanded design may be approximately 37 mm (or a different height as needed). Top surface 651a and bottom surface 651b may include a network of struts (e.g., see the struts on top surface 651a of FIG. 4C) (other networks of struts are also contemplated). Other portions of the design for the implant are also contemplated (e.g., the top half of the design shown in FIG. 1A, the bottom half of the design shown in FIG. 1A, etc). Design portions may be proportionately expanded to meet specified dimensions (e.g., specified height, width, and length). In some embodiments, the amount of struts may be reduced or material in the implant may be redistributed so that some struts may have a larger diameter and some may have a smaller diameter (e.g., the different diameters may reinforce against different directional forces). In some embodiments, a partial-design cage may be used (e.g., with half of the web structure so that the structure includes a tetrahedron. Further, in some embodiments, the implant may include angled surfaces (e.g., an angled top surface 651a and/or angled bottom surface 651b) to provide lordosis for implants to be implanted between the vertebral endplates.

In some embodiments, the web structure of an implant may distribute forces throughout the implant when implanted. For example, the connecting struts of the web structure may extend throughout the core of an implant, and the interconnectivity of struts may disperse the stress of compressive forces throughout implant to reduce the potential of stress risers (the distribution of forces throughout the implant may prevent concentration of stress on one or more portions of the vertebrae that may otherwise result in damage to the vertebrae).

In some embodiments, the web structure of an implant (e.g., the external and internal struts of the implant) may also provide surface area for bone graft fusion. For example, the web structure extending throughout an implant may add additional surface areas (e.g., on the surface of the struts making up the implant) to fuse to the bone graft material and prevent bone graft material from loosening or migrating from the implant. In some embodiments, the web structure may also support bone in-growth. For example, when implanted, adjacent bone (e.g., adjacent vertebrae if the implant is used as a spinal implant) may grow over at least a portion of struts of the implant. The bone growth and engagement between the bone growth and the implant may further stabilize the implant. In some embodiments, the surfaces of the implant may be formed with a rough surface to assist in bone in-growth adhesion.

In some embodiments, struts may have a diameter approximately in a range of about 0.025 to 5 millimeters (mm) (e.g., 1.0 mm, 1.5 mm, 3 mm, etc). Other diameters are also contemplated (e.g., greater than 5 mm). In some embodiments, the struts may have a length approximately in a range of 0.5 to 20 mm (e.g., depending on the implant size needed to, for example, fit a gap between vertebral endplates). As another example, struts may have a length approximately in a range of 30-40 mm for a hip implant. In some embodiments, the reduced strut size of the web structure may allow the open cells in implant 100 to facilitate bone growth (e.g., bone may grow through the open cells once implant 100 is implanted in the body). Average subsidence for implants may be approximately 1.5 mm within the first 3 weeks post op (other subsidence is also possible (e.g., approximately between 0.5 to 2.5 mm)). A strut size that approximately matches the subsidence (e.g., a strut size of approximately 1.5 mm in diameter and a subsidence of approximately 1.5 mm) may result in a net 0 impedance (e.g., the bone growth growing around the struts) after the implant has settled in the implanted position. The net 0 impedance throughout the entire surface area of the implant/vertebrae endplate interface may result in a larger fusion column of bone that may result in more stable fusion. Other fusion column sizes are also contemplated. The configuration of the implant may redistribute the metal throughout the implant. In some embodiments, a rim may not be included on the implant (in some embodiments, a rim may be included). The resulting bone growth (e.g., spinal column) may grow through the implant.

In some embodiments, greater than 50% of the interior volume of implant 100 may be open. In some embodiments, greater than 60%, greater than 70%, and/or greater than 80% of implant 100 may be open (e.g., 95%). In some embodiments, the open volume may be filled with bone graft material. For example, cancellous bone may be packed into an open/internal region of implant.

As the implant settles into the implant site, subsidence may place additional pressure on the bone graft material (which may already be under compressive forces in the implant) and act to push the bone graft material toward the sides of the implant (according to Boussinesq's theory of adjacent material, when a force is applied to a member that is adjacent to other materials (such as sand, dirt, or bone graft material) the force against the member creates a zone of increased pressure (e.g., 60 degrees) in the adjacent material). Struts of the implant may resist bone graft material protrusion from the sides of the web structure and may increase the pressure of the bone graft material. Bone graft material may need to be implanted in a higher-pressure environment to create an environment conducive to strong bone growth (e.g., according to Wolf's law that bone in a healthy person or animal will adapt to the loads it is placed under). The web structure may thus increase the chance of stronger fusion.

Web structures formed from other truss configurations are also contemplated. For example, the trusses may include a series of packing triangles, a two-web truss, a three-web truss, etc. Further, the web structure for an implant may include one or more trusses as described in U.S. Pat. No. 6,931,812 titled “Web Structure and Method For Making the Same”, which issued Aug. 23, 2005, which is hereby incorporated by reference in its entirety as though fully and completely set forth herein.

In some embodiments, the implant may be customized. For example, three dimensional measurements and/or shape of the implant may be used to construct an implant that distributes the web structure throughout a three-dimensional shape design.

In some embodiments, a truss/web structure may be disposed on at least a portion of an implant to facilitate coupling of the implant to an adjacent structure. For example, where an implant is implanted adjacent a bony structure, one or more truss structures may be disposed on and/or extend from a surface (e.g., an interface plate) of the implant that is intended to contact, and at least partially adhere to, the bony structure during use. In some embodiments, such as those including an intervertebral implant disposed between the end plates of two adjacent vertebrae during, one or more truss structures may be disposed on a contact surface of the intervertebral implant to facilitate bone growth that enhances coupling of the intervertebral implant to the bony structure. For example, a truss structure may include one or more struts that extend from the contact surface to define an open space for bone growth therethrough, thereby enabling bone through growth to interlock the bone structure and the truss structure with one another to couple the implant to the bony structure at or near the contact face. Such interlocking bone through growth may inhibit movement between the implant and the bony structure which could otherwise lead to loosening, migration, subsidence, or dislodging of the implant from the intended position. Similar techniques may be employed with various types of implants, including those intended to interface with tissue and/or bone structures. For example, a truss structure may be employed on a contact surface of knee implants, in a corpectomy device, in a hip replacement, in a knee replacement, in a long bone reconstruction scaffold, or in a cranio-maxillofacial implant hip implants, jaw implant, an implant for long bone reconstruction, foot and ankle implants, shoulder implants or other joint replacement implants or the like to enhance adherence of the implant to the adjacent bony structure or tissue. Examples of truss structures, and other structures, that may extend from the surface of an implant to facilitate coupling of the implant to an adjacent structure are described in U.S. Published Patent Application No. 2011/0313532, which is incorporated herein by reference. While implants described herein are depicted as being composed of substantially straight struts, it should be understood that the struts can be non-linear, including, but not limited to curved, arcuate and arch shaped. Examples of implants having non-linear struts are described in U.S. Published Patent Application No. 2013/0123935, which is incorporated herein by reference. It is known that osteoblasts under an appropriate load produce bone morphogenetic protein (“BMP”). BMPs are a group of growth factors also known as cytokines and as metabologens. BMPs act as morphogenetic signals that signal the formation of bone (i.e., an osteogenetic response). Thus, by increasing the production of one or more BMPs the osteogentic response to an implant is increased, creating an implant that is integrated into the newly formed bone.

### Device for Securing Implants to Bone Tissue

Described herein are various embodiments of implants contemplated to interface with tissue (e.g., human bone tissue) and/or bone structures in a human body. In certain embodiments, the implants described herein may be secured to tissue or bone structures to prevent undesired movement between the implant and the tissue or bone structures to which it interfaces. For instance, various devices may be implemented to enable an implant to be secured to tissue or bone structures. These devices may be made of biocompatible materials similar to those of the described implants. The devices may include various mechanisms for fastening (e.g., securing) the implant to the tissue or bone structures.

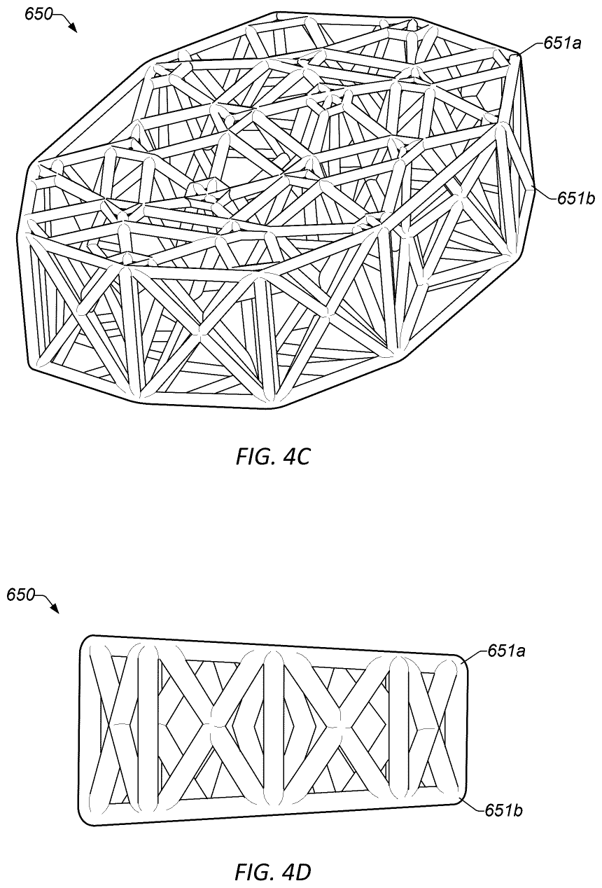

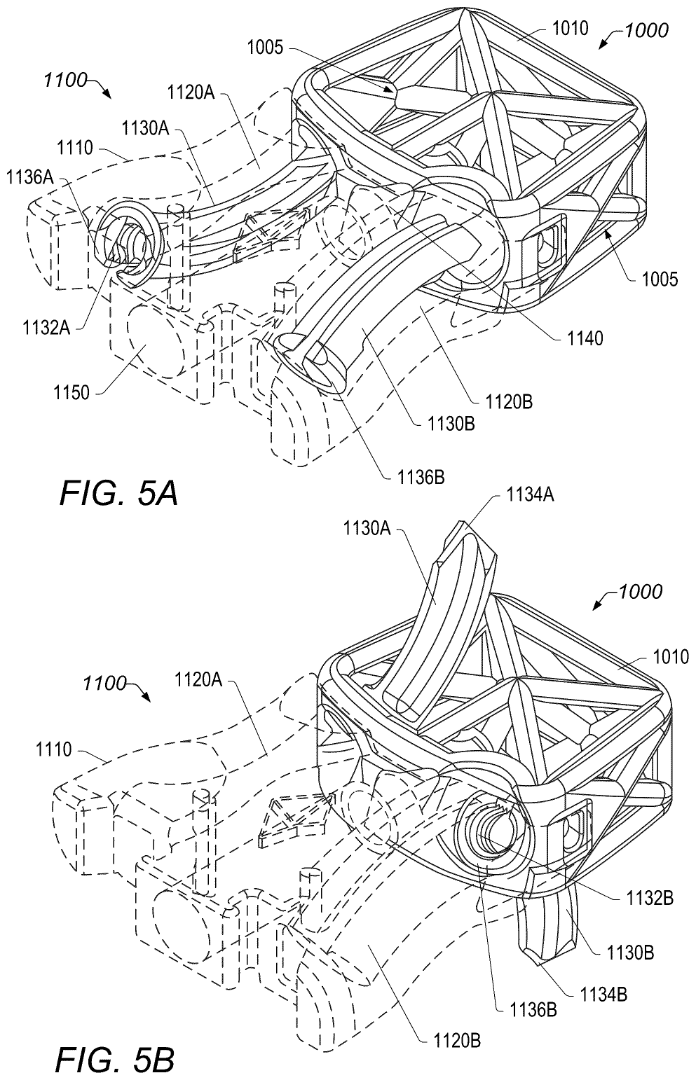

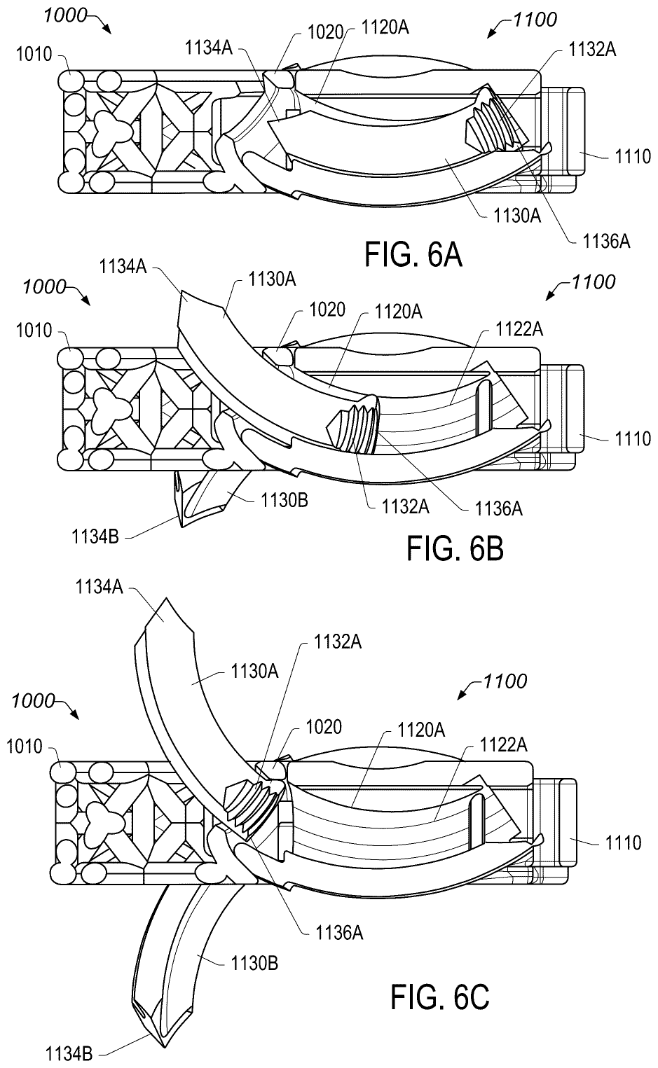

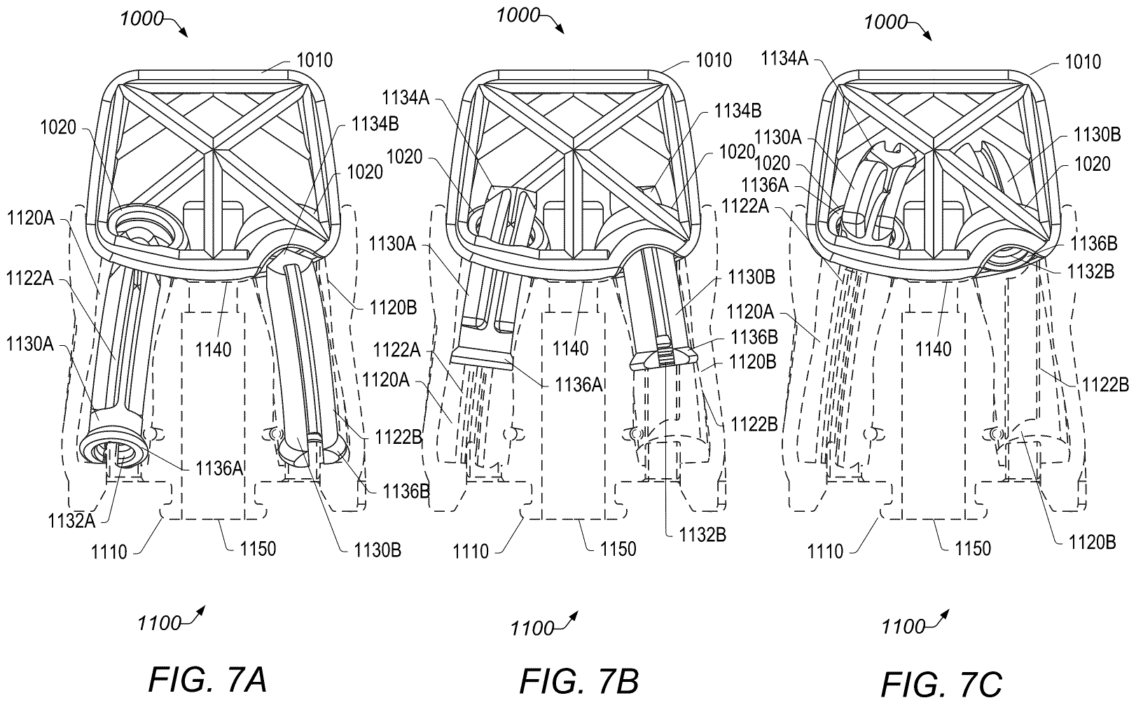

FIGS. 5-7 depict representations of a contemplated embodiment of an implant and a device for securing the implant to tissue or bone structures. FIGS. 5A and 5B depict isometric perspective view representations of a contemplated embodiment of implant 1000 and securing device 1100. FIGS. 6A-6C depict cross-sectional side view representations of the contemplated embodiment of implant 1000 and securing device 1100 in various steps of the securing process. FIGS. 7A-7C depict top view representations of the contemplated embodiment of implant 1000 and securing device 1100 in various steps of the securing process. In FIGS. 5A-5B and 7A-7C, securing device 1100 is shown with transparent outer surfaces to enable viewing of internal operations of the device. In various embodiments, implant 1000 includes one or more web structures 1005 forming implant body 1010, as shown in FIG. 5A. Web structures 1005 may include any combination of struts and nodes described herein.

In the illustrated embodiment of FIGS. 5-7, device 1100 includes guide body 1110, anchor pathways 1120, and anchor devices 1130. In certain embodiments, device 1100 includes two anchor pathways 1120A, 1120B corresponding to two anchor devices 1130A, 1130B. In various embodiments, guide body 1110 is coupled to (e.g., secured to or attached to) implant 1000 using fastener 1140. Fastener 1140 may be, for example, a threaded fastener such as a screw. Fastener 1140 may include other structures for fastening the fastener to implant 1000. Fastener 1140 may be inserted through hole 1150 in guide body 1110 and coupled to an attachment point on implant 1000. In some embodiments, the attachment point on implant 1000 may have threads or other structures configured to mate with threads or structures on fastener 1140.

After device 1100 is coupled to implant 1000, the device and implant may be positioned to interface with human tissue (e.g., human bone tissue) and/or bone structures in a human body. Anchor devices 1130 in device 1100 may then be “activated” (e.g., extended into the tissue) to secure implant 1000 to the human tissue, as shown in FIGS. 5-7. FIGS. 5A, 6A, and 7A depict device 1100 with anchor devices 1130 in an “implant installation position”. In the implant installation position, anchor devices 1130 are retracted inside anchor pathways 1120 and the perimeter of guide body 1110 to allow device 1100 and implant 1000 to be positioned to interface with tissue. For instance, implant 1000 and device 1100 may be placed in contact with the tissue. In some instances, temporary or impermanent fasteners may be used to hold implant 1000 and/or device 1100 in contact with the tissue while device 1100 is activated for further engagement with the tissue.

After device 1100 and implant 1000 are interfaced with the tissue, anchor devices 1130 may be “activated” to engage the tissue interfaced with the implant. For instance, anchor devices 1130 may undergo an activation process to move the anchor devices 1130 along anchor pathways 1120, through implant body 1010, and to engage (e.g., couple to or attach to) the tissue interfacing with the implant body. The movement of anchor device 1130 during the activation process is shown in FIGS. 5-7. The activation process begins from the implant installation position shown in FIGS. 5A, 6A, and 7A. An intermediate step (e.g., a “halfway” step) of the activation process is shown in FIGS. 6B and 7B while a final position (e.g., “implant securement position”) is shown in FIGS. 5B, 6C, and 7C. In the final position, anchor devices 130 are engaged with and secure implant body 1010 to the tissue interfacing with the implant body. While FIGS. 5-7 depict simultaneous movement of anchor device 1130A and anchor device 1130B, it should be understood that the anchor devices may be moved independently and/or at different times (e.g., anchor device 1130A may be activated first and then anchor device 1130B is activate subsequent to anchor device 1130A completing engagement with tissue or the anchor devices may be alternatingly activated in incremental steps).

In certain embodiments, anchor devices 1130 are activated by moving (e.g., pushing) the anchor devices within anchor pathways 1120 towards implant body 1010. In some embodiments, anchor devices 1130 may be moved by engaging an activation device (e.g., driver) with threads 1132 (e.g., threads 1132A and 1132B, shown in FIGS. 5-7) in the anchor devices. For instance, a threaded activation device (such as a screw or other threaded device) may engage threads 1132 and be used to drive anchor devices 1130 into and through implant body 1010. The activation device may be a single activation device that engages both threads 1132A and threads 1132B simultaneously or separate activation devices may be used for each of the threads. Alternatively, a single activation device may be used to activate threads 1132A first and then subsequently used to activate threads 1132B, or vice versa. In some embodiments, anchor pathways 1120 may include rails 1122 (e.g., rails 1122A, 1122B) that guide movement of anchor devices 1130 along the anchor pathways. Rails 1122 may, for example, inhibit rotation of anchor devices 1130 while the anchor devices are driven along anchor pathways 1120 (e.g., moved by the activation device(s)).

As shown in FIGS. 6B and 7B, as anchor devices 1130 move along anchor pathways 1120, insertion ends 1134 (e.g., insertion ends 1134A and 1134B) of the anchor devices move into implant body 1010 through a side surface of the implant body. Insertion ends 1134 then move through implant body 1010 and begin to protrude out of the upper and lower surfaces of the implant body (e.g., surfaces perpendicular to the surface of entry of the insertion ends). For example, as shown in FIG. 6B, insertion end 1134A is protruding out of the upper surface of implant body 1010 and insertion end 1134B is protruding out of the lower surface of the implant body 1010. In some embodiments, the upper/lower surfaces of implant body 1010 include openings that allow anchor devices 1130 to move through the surfaces. As insertion end 1134A and insertion end 1134B protrude out of the upper/lower surfaces of implant body 1010, the insertion ends will begin to engage tissue interfacing with the implant body. In certain embodiments, insertion ends 1134A, 1134B have pointed ends (or other shapes) that allow anchor devices 1130 to penetrate the tissue. For instance, anchor device 1130 may be nails, or similar fasteners, with pointed insertion ends 1134.

Activation of anchor devices 1130 may continue until the anchor devices complete their travel along anchor pathways 1120 and are in the final position and fully engaged with tissue interfacing implant body 1010. The final position of anchor devices 1130 in the fully engaged position with tissue is shown in FIGS. 5B, 6C, and 7C. In the final position of anchor devices 1130, insertion ends 1134 have fully extended out of the upper/lower surfaces of implant body 1010 and are fully engaged with tissue interfacing with the implant body. With anchor devices 1130 fully engaged with the tissue interfacing with implant body 1010, implant 1000 (along with securing device 1100) is secured to the tissue. For instance, insertion ends 1134 are secured to the tissue while trailing ends 1136 (e.g., ends 1136A, 1136B of anchor devices 1130A, 1130B, respectively) are secured to implant body 1010. Trailing ends 1136 may, for example, engage surfaces 1020 (shown in FIGS. 6C and 7C) in implant body 1010 such that anchor devices 1130 “pull” the implant body towards the tissue when the anchor devices are fully inserted to and engaged with the tissue.

Securing implant 1000 to the tissue may inhibit undesirable movement of the implant with respect to the tissue. In some contemplated embodiments, securing device 1100 may be uncoupled from implant 1000 after the implant is secured to the tissue by anchor devices 1130 and then the securing device can be removed from the human body and discarded.

In various embodiments, as shown in FIGS. 5-7, anchor devices 1130 may have an arcuate path of travel. For example, anchor pathways 1120 and anchor devices 1130 may have similar arcs. With the similar arcs, anchor pathways 1120 may directionally guide anchor devices 1130 as the anchor devices move along the anchor pathways. The arcuate path of travel for anchor devices 1130 may provide secure engagement with tissue interfacing implant body 1010. For example, insertion ends 1134 may arc into the tissue such that the insertion ends “grab” tissue as they move into the tissue. Grabbing the tissue may be a more secure engagement between anchor devices 1130 and the tissue than merely contact between the anchor devices and the tissue.

Additionally, the travel path for anchor devices 1130 provided by anchor pathways 1120 may allow guide body 1110 to be coupled to implant body 1010 along a same plane such that securing device 1100 has a similar profile to implant 1000 and the securing device and the implant have at least one continuous surface engaging the tissue. Both implant 1000 and securing device 1100 having a similar profile allows accurate placement of the group of devices together in the tissue. For example, both implant 1000 and securing device 1100 devices may together be placed flat on a section of bone tissue with the devices having one or more continuous surfaces engaging the tissue.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An implant system for interfacing with a bone structure, comprising:
an implant body configured to interface with human bone tissue;
a guide body coupled to at least one surface of the implant body, one or more anchor pathways being defined in the guide body; and
one or more anchor devices positioned in the anchor pathways of the guide body, wherein first ends of the anchor devices are configured to be moved through at least a portion of the implant body to insert into at least a portion of the human bone tissue, the anchor pathways directionally guiding the anchor devices through the implant body and into the human bone tissue, wherein, when the first ends of the anchor devices are inserted into the human bone tissue, the anchor devices secure the implant body to the human bone tissue.

2. The implant of claim 1, wherein the implant body has one or more web structures.

3. The implant of claim 2, wherein the web structures include a plurality of struts joined at nodes.

4. The implant of claim 2, wherein the implant body comprises openings in the web structures for allowing the anchor devices to move through the implant body.

5. The implant of claim 1, wherein the implant body has a rectangular structure, and wherein the guide body is coupled to a side of the implant body.

6. The implant of claim 1, wherein the anchor devices enter the implant body through the at least one surface and exit the implant body through one or more additional surfaces of the implant body.

7. The implant of claim 1, wherein the anchor devices include a first anchor device and a second anchor device, and wherein the first anchor device and the second anchor device exit the implant body through opposing surfaces of the implant body.

8. The implant of claim 1, wherein the anchor devices include a first anchor device and a second anchor device, wherein the first anchor device enters the implant body through the at least one surface and exits the implant body through a first additional surface of the implant body perpendicular to the at least one surface, and wherein the second anchor device enters the implant body through the at least one surface and exits the implant body through a second additional surface of the implant body perpendicular to the at least one surface, the second additional surface being opposite the first additional surface.

9. The implant of claim 1, wherein the guide body is coupled to the implant body such that the bodies have at least one continuous surface for interfacing with the human bone tissue.

10. The implant of claim 1, wherein the anchor pathways are arcuate paths through the guide body.

11. The implant of claim 1, wherein the anchor pathways guide the anchor devices into the implant body through the at least one surface and out of the implant body through additional surfaces of the implant body perpendicular to the at least one surface.

12. The implant of claim 1, wherein the anchor devices include a first anchor device and a second anchor device, and wherein the anchor pathways guide the first anchor device and the second anchor device to exit the implant body through opposing surfaces of the implant body.

13. The implant of claim 1, wherein the anchor pathways include rails for guiding movement of the anchor devices through the guide body.

14. The implant of claim 1, wherein the anchor devices comprise nails configured to secure the implant to the human bone tissue when inserted into the human bone tissue.

15. The implant of claim 1, wherein the first ends of the anchor devices comprise shapes configured for penetrating the human bone tissue.

16. The implant of claim 1, wherein second ends of the anchor devices comprise means for receiving a driver configured to move the anchor devices along the anchor pathways.

17. The implant of claim 1, wherein the first ends of the anchor devices are proximal to the implant body when the guide body is coupled to the implant body.

18. The implant of claim 1, wherein the guide body is coupled to the implant body using at least one fastener.

19. An implant system for interfacing with a bone structure, comprising:
an implant body having one or more web structures, wherein the implant body is configured to interface with human bone tissue;
a guide body coupled to at least one surface of the implant body, one or more anchor pathways being defined in the guide body with first ends and second ends, the second ends being at an interface between the guide body and the implant body; and
one or more anchor devices positioned in the anchor pathways of the guide body, wherein first ends of the anchor devices are configured to be moved from the first ends of the anchor pathways to the second ends of the anchor pathways such that second ends of the anchor devices move through a portion of the implant body and insert into at least a portion of the human bone tissue, the anchor pathways directionally guiding the anchor devices through the implant body and into the human bone tissue, wherein, when the second ends of the anchor devices are inserted into the human bone tissue, the anchor devices secure the implant body to the human bone tissue.

20. The implant of claim 19, wherein at least two of the anchor pathways guide at least two anchor devices to exit the implant body through at least two opposing surfaces of the implant body.
